(19) United States
(12) Patent Application Publication
(10) Pub. No.: US 2018/0200175 A1
(43) Pub. Date: Jul. 19, 2018

(54) HAND POWDER COMPOSITION PROVIDING ENHANCED SLIP OR GLIDE FEEL

(71) Applicant: David Dunwoodie, Delta (CA)

(72) Inventor: David Dunwoodie, Delta (CA)

(21) Appl. No.: 15/873,284

(22) Filed: Jan. 17, 2018

Related U.S. Application Data
(60) Provisional application No. 62/447,175, filed on Jan. 17, 2017.

(51) Int. Cl. A61K 8/69; A61K 8/19; A61K 8/02; G10D 3/00

(52) U.S. Cl. CPC A61K 8/69; A61K 8/19; C08L 27/18; G10D 3/00; C08K 2201/005; A61K 8/022

(57) ABSTRACT

A hand powder composition, particularly useful for musicians of necked stringed instruments, comprising a fine moisture absorbing powder in an amount from about 90%-99% by weight of the total composition and a fine polytetrafluoroethylene powder in an amount from about 1%-7% by weight of the total composition. The polytetrafluoroethylene powder may have average particle sizes of about 18-22 μm. The moisture absorbing powder may be talcum powder or corn starch, or a combination thereof. A fine alkaline powder may be included to neutralize acidity of perspiration. A fine powdered colorant may be included for approximately matching the color of the composition to a desired color.

### BACKGROUND OF THE INVENTION FIELD OF THE INVENTION

The present invention relates generally to hand powder compositions for providing enhanced slip or glide feel, in particular to hand powder compositions for use by stringed instrument musicians that provides moisture absorbency and enhanced slip and glide feel when playing necked stringed instruments such as guitars, banjos, ukuleles and the like.

### BACKGROUND OF THE INVENTION

In the field of necked stringed musical instruments, the musician plays the strings with one hand while moving his or her other hand along the neck and strings of the instrument to position the fingers so that they may depress the played strings against the neck to produce the desired note or notes. The tempo of the music and the location along the neck of the desired notes may require that the musician to rapidly slide his or hand back and forth along the neck and strings of the instrument. The smooth sliding of the hand along the neck of the instrument is often hindered by perspiration on the musician's palm and fingers. Perspiration or wetness on the hands increases in hot, humid climates or locations. And the amount that the musician perspires is often exacerbated during highly demanding performances, such as concerts, due to one or a combination of the musician's anxiety, the ambient temperature and humidity of the venue and the level of the musician's physical exertion. There is a further problem that the acidity of the perspiration on the musician's hand often contributes to corrosion of the strings and other metal components of the instrument.

In order to deal with the hindrance caused by perspiration on one's hand, some musicians have tried using commercially available baby powders made of either talcum powder or cornstarch as a means of absorbing some of the perspiration. This may work to some extent in individuals who tend to perspire less and during performances involving a low degree of physical exertion. However, a problem with baby powders made of talc is that the feel of the talc powder on the hands is chalky and gritty against the instrument neck and strings when played, and talc powder has a tendency to cake on the instrument, particularly on the sides of the fretboard. A problem with baby powders made of cornstarch or other starches is that they become pasty when combined with excessive moisture, which actually hinders the gliding motion of the musician's hand and fingers along the instrument neck and strings during instances of heavier perspiration, detracting from the playing experience, and leaves a lot of residue on the neck and strings.

There is a need for an improved hand powder composition for musicians of necked stringed instruments that provides moisture absorbency to alleviate the problem of perspiration but which also provides a smooth slip or gliding feel on the neck and strings as the musician's hand moves up and down the neck while playing the instrument.

### SUMMARY OF THE INVENTION

The inventor surprisingly discovered that a hand powder composition comprising of a relatively small amount of fine polytetrafluoroethylene powder and a fine water absorbing powder, such as baby powder made from talc or cornstarch, provides a smooth feel and enhanced glide of the hand and fingers over the neck and strings of an instrument, without the problem of gumming up or becoming pasty due to perspiration. Furthermore, the inclusion of an alkaline powder, such as sodium bicarbonate, in the hand powder composition in a very small relative amount, helps neutralize or pH balance the acidity of perspiration on the hands, which reduces the corrosive effect that such perspiration would otherwise have on the components of the musical instrument.

Accordingly, in some aspects, the present invention provides a hand powder composition for enhanced slip or glide feel, particularly useful for musicians of necked stringed instruments such as for example guitars, banjos, ukuleles, and the like, but also useful for other applications requiring enhanced slip or glide feel between a hand and an object such as when playing billiards, the hand powder comprising: a fine moisture absorbing powder in an amount from about 90%-99% by weight of the total composition; and a fine polytetrafluoroethylene powder in an amount from about 1%-7% by weight of the total composition. Preferably, the polytetrafluoroethylene powder has average particle sizes of about 18-22 μm.

In some embodiments, the moisture absorbing powder is talcum powder, or corn starch, or a combination thereof.

In some embodiments, the amount of polytetrafluoroethylene is from about 2%-4% by weight of the composition. In some embodiments, the amount of polytetrafluoroethylene is about 3.5% by weight of the composition.

In some embodiments, the hand powder composition may further comprise a fine alkaline powder to neutralize acidity of perspiration. In some embodiments, the alkaline powder is sodium bicarbonate in an amount from about 0.25%-1.5% by weight of the composition. In some embodiments, the amount of sodium bicarbonate is about 1% by weight of the composition.

In some embodiments, the hand powder composition may further comprise a fine powdered colorant for approximately matching the color of the composition to a desired color.

### DETAILED DESCRIPTION

The present invention provides hand powder compositions that provide a combination of desirable properties including moisture absorbency and enhanced slip and glide feel, making it especially useful for musicians playing necked stringed instruments such as guitars, banjos, ukuleles and the like. The hand powder compositions may be used for other applications requiring enhanced slip or glide feel between a hand and an object, such as for example between the hand of a billiard player and the billiard cue in the game of billiards.

The hand powder composition comprises a fine moisture absorbing powder making up about 90%-99% of the composition by weight, and a fine polytetrafluoroethylene powder making up about 1%-7% of the composition by weight.

The moisture absorbing powder may be a finely ground starch, such as for example corn starch of the kind used in baby powders. Preferably the moisture absorbing powder is a fine talcum powder such as the kind used in baby powders. The particle size distributions of these finely ground or fine powders are typically such that 100% passes through a 60 mesh screen and not less than 99% passes through a 100 mesh screen and at least 98% passes through a 200 mesh screen.

The talc that is useful in the present invention is a cosmetic grade of talc, which is a white, odorless, fine powder ground from a naturally occurring rock ore and it typically consists of about 90% hydrous magnesium silicate having a structural formula of Mg₃ (Si₄.O₁₀).(OH)₂, with the remainder consisting of naturally associated minerals such as calcite, chlorite, dolomite, kaolin, and magnesite and containing no asbestos minerals.

Preferably, the polytetrafluoroethylene powder is a cosmetic grade fine powder having average particle sizes of about 18-22 μm as these have been found to provide a desired slip or glide feel. An example of a preferable polytetrafluoroethylene powder for use in the present invention is a product sold as FluoroPURE™ 8200 by Presperse of N.J., USA. Preferably, the polytetrafluoroethylene powder is present in an amount from about 1% to about 7% of the composition by weight, and more preferably from about 2% to about 4% of the composition by weight, While higher percentages of polytetrafluoroethylene may be used, doing so increases cost and decreases the overall moisture absorbency of the composition.

The hand powder composition may further include a fine alkaline powder to neutralize acidity of perspiration. Preferably, the alkaline powder is sodium bicarbonate in an amount from about 0.25%-1.5% of the composition by weight.

In some embodiments, a fine powdered colorant may be added to the composition to achieve a desired color of the overall hand powder composition. For example, the composition may include powdered colorants to approximate a skin tone to make it less noticeable to an audience that the musician is using the hand powder composition.

An example of a hand powder composition comprising about 95.5% talcum powder, about 3.5% polytetrafluoroethylene powder (FluoroPURE™ 8200) and about 1% sodium bicarbonate powder, by weight of total composition, has been found to provide the desired advantages of moisture absorbency, superior slip and glide feel of the hand and fingers over the neck and strings of the instrument, and pH balance to reduce the corrosive effect of perspiration on the metal components on the instrument.

While specific embodiments of the invention have been described, such embodiments are illustrative of the invention only and should not be taken as limiting its scope. In light of the present disclosure, many modifications will occur to those skilled in the art to which the invention relates, and the invention, therefore, should be construed in accordance with the accompanying claims.

What is claimed is:

1. A hand powder composition comprising: a fine moisture absorbing powder in an amount from about 90%-99% by weight of the total composition; and
a fine polytetrafluoroethylene powder in an amount from about 1%-7% by weight of the total composition.

2. The hand powder composition of claim 1 wherein the average particle sizes of the polytetrafluoroethylene powder are about 18-22 μm.

3. The hand powder composition of any one of claims 1-2 wherein the wherein the moisture absorbing powder is talcum powder.

4. The hand powder composition of any one of claims 1-2 wherein the moisture absorbing powder is corn starch.

5. The hand powder composition of any one of claims 1-4 wherein the amount of polytetrafluoroethylene is from about 2%-4% by weight of the composition.

6. The hand powder composition of any one of claims 1-4 wherein the amount of polytetrafluoroethylene is about 3.5% by weight of the composition.

7. The hand powder composition of any one of claims 1-6 further comprising a fine alkaline powder to neutralize acidity of perspiration.

8. The hand powder composition of claims 7 wherein the alkaline powder is sodium bicarbonate in an amount from about 0.25%-1.5% by weight of the composition.

9. The hand powder composition of claims 8 wherein the amount of sodium bicarbonate is about 1% by weight of the composition.

10. The hand powder composition of any one of claims 1-9 further comprising a fine powdered colorant for approximately matching the color of the composition to a desired color.

11. The use of a hand powder composition of any one of claims 1-10 for playing necked stringed musical instruments.

12. The use of a hand powder composition of any one of claims 1-10 on a hand for increasing glide feel between the hand and a neck of a stringed musical instrument.

13. The use of a hand powder composition of any one of claims 1-10 for playing billiards.

14. The use of a hand powder composition of any one of claims 1-10 on a hand for increasing glide feel between the hand and a billiard cue.
